(19) United States
(12) Patent Application Publication
(10) Pub. No.: US 2013/0059872 A1
(43) Pub. Date: Mar. 7, 2013

(54) POLYMORPHS OF ALOGLIPTIN BENZOATE

(71) Applicants: Ehud Marom, Kfar Saba (IL); Shai Rubnov, Tel Aviv (IL)

(72) Inventors: Ehud Marom, Kfar Saba (IL); Shai Rubnov, Tel Aviv (IL)

(73) Assignee: MAPI PHARMA LIMITED, Ness Ziona (IL)

(21) Appl. No.: 13/697,455

(22) PCT Filed: Nov. 22, 2010
(86) PCT No.: PCT/IL10/00974

§ 371 Date: Nov. 12, 2012

(30) Foreign Application Priority Data
May 12, 2010 (IL) PCT/IL2010/000378

(51) Int. Cl. C07D 401/04; A61P 3/10; A61K 31/513

(52) U.S. Cl. 514/274; 544/312

(57) ABSTRACT

The present invention provides new amorphous forms of alogliptin benzoate, pharmaceutical compositions comprising same, methods for their preparation and use thereof in treating conditions mediated by DPP-IV, in particular, type 2 diabetes.

### FIELD OF THE INVENTION

The present invention relates to new forms of alogliptin benzoate, pharmaceutical compositions comprising same, and use thereof in treating type 2 diabetes.

### BACKGROUND OF THE INVENTION

Alogliptin is a selective serine protease dipeptidyl-peptidase IV (DPP IV) inhibitor effective in maintaining glucose homeostasis by controlling the incretin activity of glucagon-like peptide 1 (GLP-1) and glucose-dependent insulinotropic polypeptide (GIP, also known as gastric inhibitory polypeptide). It has thus been suggested as a potent drug for the treatment of type 2 diabetes. The benzoate salt of alogliptin (SYR-322) has demonstrated encouraging antidiabetic efficacy.

Alogliptin is chemically named 2-[6-[3(R)-Aminopiperidin-1-yl]-3-methyl-2,4-dioxo-1,2,3,4-tetrahydropyrimidin-1-ylmethyl]benzonitrile, and is represented by the following chemical structure:

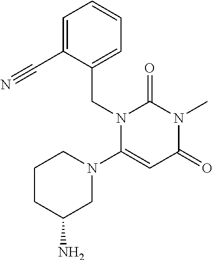 [SMILES: Cn1c(=O)cc(N2CCC[C@@H](N)C2)n(Cc2ccccc2C#N)c1=O]

Alogliptin and salts thereof and processes for their preparation are disclosed in EP 1586571 (WO 2005/095381); WO 2008/067465; WO 2007/035379, US 2004/0097510, and in WO 2010/109468 to some of the inventors of the present invention.

A new form of a compound may possess physical properties that differ from, and are advantageous over, those of other crystalline or amorphous forms. These include, packing properties such as molar volume, density and hygroscopicity; thermodynamic properties such as melting temperature, vapor pressure and solubility; kinetic properties such as dissolution rate and stability under various storage conditions; surface properties such as surface area, wettability, interfacial tension and shape; mechanical properties such as hardness, tensile strength, compactibility, handling, flow and blend; and filtration properties. Variations in any one of these properties affect the chemical and pharmaceutical processing of a compound as well as its bioavailability and may often render the new form advantageous for medical use.

US 2007/0066636 discloses polymorphs of the tartrate salt of alogliptin, compositions, kits and articles of manufacture comprising said polymorphs, and methods of their use.

WO 2007/035372 (US 2007/0066635) discloses two polymorphs of alogliptin benzoate, a crystalline polymorph and an amorphous polymorph, designated as Form A and Form 1, respectively. The crystalline form was characterized by the following distinguishing X-ray diffraction peaks at 9.44, 10.84, 17.82, 18.75, 25.87 and 28.52 2θ°. The amorphous form was characterized by an X-ray powder diffraction pattern that shows a broad halo with no specific peaks present. The amorphous form was further characterized by an IR spectrum comprising unique peaks at 809, 868, 1119, 1599 and 1703 cm⁻¹; an FT-Raman spectrum with unique peak positions at 805, 1280 and 1703 cm⁻¹; a differential scanning calorimetry (cyclic DSC) spectrum having a Tg=70° C. (onset), an exotherm at 132° C. (maxima), and an endotherm at 183° C. (onset temperature); and a thermogravimetric analysis data showing a 4% weight loss from 25-151° C.

There remains an unmet need for additional solid state forms of alogliptin or salts thereof having good physiochemical properties, desirable bioavailability, and advantageous pharmaceutical parameters.

### SUMMARY OF THE INVENTION

The present invention provides new amorphous forms of alogliptin benzoate, pharmaceutical compositions comprising said forms, methods for their preparation and use thereof in treating conditions mediated by DPP-IV and, in particular, type 2 diabetes.

The present invention is based in part on the unexpected finding that the new amorphous forms disclosed herein possess advantageous physicochemical properties which render their processing as medicaments beneficial. The forms of the present invention have good bioavailability as well as desirable stability characteristics enabling their incorporation into a variety of different formulations particularly suitable for pharmaceutical utility.

According to one aspect, the present invention provides an amorphous form of alogliptin benzoate (form I) characterized by a DSC profile substantially as shown in FIG. 2. In one embodiment, the amorphous form I of alogliptin benzoate has a glass transition temperature between about 66° C. and about 77° C. In another embodiment, the amorphous form I of alogliptin benzoate is characterized by a TGA profile substantially as shown in FIG. 4. In yet another embodiment, the amorphous form I is characterized by an IR spectrum substantially as shown in FIG. 5. In other embodiments, the amorphous form I of alogliptin benzoate has an IR spectrum with characteristic peaks at about 401±4, 448±4, 525±4, 559±4, 586±4, 608±4, 672±4, 722±4, 766±4, 805±4, 832±4, 864±4, 948±4, 964±4, 1024±4, 1066±4, 1167±4, 1225±4, 1285±4, 1376±4, 1438±4, 1549±4, 1652±4, 1701±4, 2224±4, 2852±4, 2947±4, 3064±4, and 3292±4 cm⁻¹. In certain embodiments, the amorphous form I of alogliptin benzoate is characterized by a Raman spectrum substantially as shown in FIG. 6. In particular embodiments, the Raman spectrum of amorphous alogliptin benzoate form I has characteristic peaks at about 194±4, 237±4, 289±4, 319±4, 348±4, 396±4, 415±4, 470±4, 534±4, 593±4, 672±4, 745±4, 767±4, 811±4, 848±4, 917±4, 945±4, 1004±4, 1045±4, 1087±4, 1111±4, 1170±4, 1186±4, 1274±4, 1293±4, 1379±4, 1468±4, 1486±4, 1565±4, 1602±4, 1654±4, 1697±4, 1748±4, 1770±4, 1863±4, 2229±4, and 2950±4 cm⁻¹.

In one embodiment, the present invention provides a process for preparing amorphous form I of alogliptin benzoate, the process comprising the steps of:

(a) heating a alogliptin benzoate, preferably alogliptin benzoate Form A to melt; and

(b) cooling the melted alogliptin benzoate obtained in step (a), so as to obtain amorphous alogliptin benzoate Form I.

In some embodiments, the cooling in step (b) is selected from fast cooling and slow cooling. Each possibility represents a separate embodiment of the invention.

According to another aspect, the present invention provides an amorphous form of alogliptin benzoate (form II) characterized by a Raman spectrum substantially as shown in FIG. 12. In particular embodiments, the Raman spectrum of amorphous alogliptin benzoate form II has characteristic peaks at about 94±4, 125±4, 155±4, 195±4, 234±4, 449±4, 537±4, 597±4, 682±4, 718±4, 745±4, 816±4, 843±4, 857±4, 915±4, 945±4, 1004±4, 1060±4, 1087±4, 1119±4, 1177±4, 1248±4, 1273±4, 1290±4, 1364±4, 1387±4, 1466±4, 1483±4, 1529±4, 1570±4, 1654±4, 1685±4, 1744±4, 1769±4, 1786±4, 1847±4, 1876±4, 1904±4, 2950±4, 3007±4 and 3039±4 cm⁻¹. In certain embodiments, the amorphous form II of alogliptin benzoate is characterized by a DSC profile substantially as shown in FIG. 8. In some embodiments, the amorphous form II of alogliptin benzoate has a glass transition temperature between about 68° C. and about 73° C. In other embodiments, the amorphous form II of alogliptin benzoate is characterized by a TGA profile substantially as shown in FIG. 10. In yet other embodiments, the amorphous form II is characterized by an IR spectrum substantially as shown in FIG. 11. In some embodiments, the amorphous form II of alogliptin benzoate has an IR spectrum with characteristic peaks at about 405±4, 521±4, 558±4, 600±4, 604±4, 673±4, 695±4, 722±4, 766±4, 810±4, 833±4, 866±4, 948±4, 1024±4, 1067±4, 1133±4, 1172±4, 1228±4, 1376±4, 1441±4, 1558±4, 1655±4, 1705±4, 2224±4, 2848±4, 2951±4, and 3052±4 cm⁻¹.

In some embodiments, the present invention provides a process for preparing amorphous form II of alogliptin benzoate, the process comprising the steps of:
(a) dissolving alogliptin benzoate, preferably alogliptin benzoate Form A in ethanol; and
(b) evaporating the solvent to precipitate amorphous alogliptin benzoate form II.

In certain embodiments, the present invention provides a pharmaceutical composition comprising as an active ingredient any one of the amorphous alogliptin benzoate forms of the present invention, and a pharmaceutically acceptable carrier. In one embodiment, the composition comprises the amorphous alogliptin benzoate form I described in the present application. In another embodiment, the composition comprises the amorphous alogliptin benzoate form II described in the present application.

In a particular embodiment, the pharmaceutical composition is in the form of a tablet.

In various embodiments, the present invention provides a pharmaceutical composition comprising as an active ingredient any one of the amorphous alogliptin benzoate forms of the present invention, and a pharmaceutically acceptable carrier for use in treating a condition mediated by DPP-IV. In one embodiment, the composition comprises the amorphous alogliptin benzoate form I described in the present application. In another embodiment, the composition comprises the amorphous alogliptin benzoate form II described in the present application.

In particular embodiments, the condition mediated by DPP-IV is type 2 diabetes.

In some embodiments, the present invention provides a method of treating a condition mediated by DPP-IV comprising administering to a subject in need thereof an effective amount of a composition comprising any one of the amorphous alogliptin benzoate forms of the present invention. In one embodiment, the composition comprises the amorphous alogliptin benzoate form I described in the present application. In another embodiment, the composition comprises the amorphous alogliptin benzoate form II described in the present application.

In additional embodiments, the present invention provides the use of any one of the amorphous alogliptin benzoate forms of the present invention for the preparation of a medicament for treating a condition mediated by DPP-IV. In one embodiment, the amorphous alogliptin benzoate is a form I amorphous alogliptin benzoate as described in the present application. In another embodiment, the amorphous alogliptin benzoate is a form II amorphous alogliptin benzoate as described in the present application.

In particular embodiments, the method and use disclosed herein are designated for treating type 2 diabetes.

In some embodiments, the subject is a mammal, preferably a human.

Further embodiments and the full scope of applicability of the present invention will become apparent from the detailed description given hereinafter. However, it should be understood that the detailed description and non-limiting examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 illustrates characteristic X-ray diffraction patterns of amorphous Form I of alogliptin benzoate, obtained by fast (panel A) or slow (panel B) cooling under vacuum. Also shown for comparison is the X-ray diffraction pattern of alogliptin benzoate Form A of WO 2007/035372 (panel C).

FIG. 2 illustrates a characteristic Differential Scanning Calorimetry (DSC) profile of amorphous form I of alogliptin benzoate.

FIG. 3 illustrates a characteristic Modulate DSC profile of amorphous form I of alogliptin benzoate.

FIG. 4 illustrates a characteristic Thermogravimetric analysis (TGA) profile of amorphous form I of alogliptin benzoate.

FIG. 5 illustrates a characteristic Infrared (IR) spectrum of amorphous form I of alogliptin benzoate.

FIG. 6 illustrates a characteristic Fourier Transform-Raman (FT-Raman) spectrum of amorphous form I of alogliptin benzoate.

FIG. 7 illustrates characteristic X-ray diffraction patterns of amorphous Form II of alogliptin benzoate, obtained by fast precipitation from a saturated solution of EtOH (panel C). Also shown for comparison are the X-ray diffraction patterns of alogliptin benzoate Form A of WO 2007/035372 (panel D) and two additional amorphous forms obtained by fast precipitation from saturated solutions of DCM (panel A) and acetone (panel B).

FIG. 8 illustrates a characteristic Differential Scanning Calorimetry (DSC) profile of amorphous form II of alogliptin benzoate.

FIG. 9 illustrates a characteristic Modulate DSC profile of amorphous form II of alogliptin benzoate.

FIG. 10 illustrates a characteristic Thermogravimetric analysis (TGA) profile of amorphous form II of alogliptin benzoate.

FIG. 11 illustrates a characteristic Infrared (IR) spectrum of amorphous form II of alogliptin benzoate.

FIG. 12 illustrates a characteristic Fourier Transform-Raman (FT-Raman) spectrum of amorphous form II of alogliptin benzoate.

### DETAILED DESCRIPTION OF THE INVENTION

The present invention is directed to novel amorphous forms of 2-[6-[3(R)-Aminopiperidin-1-yl]-3-methyl-2,4-dioxo-1,2,3,4-tetrahydropyrimidin-1-ylmethyl]benzonitrile benzoate.

The present invention is further directed to pharmaceutical compositions comprising the amorphous forms and a pharmaceutically acceptable carrier and their use in treating conditions mediated by DPP-IV.

The present invention is further directed to methods of preparing the novel amorphous forms of alogliptin benzoate.

Polymorphs are two or more solid state phases of the same chemical compound that possess different arrangement and/or conformation of the molecules. Polyamorphism is the ability of a substance to exist in several different amorphous forms. Different forms of amorphous pharmaceuticals with readily discernible physical and chemical characteristics and some marked differences in their pharmaceutical performance have been reported. Even though amorphous materials do not exhibit long-range periodic atomic ordering, different amorphous phases of the same chemical substance can exhibit significant structural differences in their short-range atomic arrangement. These differences may lead to different physical and chemical properties such as density, stability, processability, dissolution and even bioavailability. Polyamorphism in pharmaceuticals is reviewed in Hancock et al. (Journal of Pharmacy and Pharmacology 2002, 54: 1151-1152), the content of which is hereby incorporated by reference.

One important physical property of a compound used as an active ingredient of a medicament is its stability at ambient conditions, especially to moisture, and under storage conditions. The identification and characterization of various polymorphic forms, e.g., amorphous forms of a pharmaceutically active compound is therefore of great significance in obtaining medicaments with desired properties including a characteristic dissolution rate, milling property, bulk density, thermal stability or shelf-life. The amorphous alogliptin benzoate forms of the present invention possess improved characteristics of hygroscopicity, bulk density and solubility in aqueous media. Furthermore, the amorphous alogliptin benzoate forms of the present invention have improved chemical and solid state stability as is evident from their thermal analysis profiles. Hence, these forms may be more stable when stored over prolonged periods of time.

In one embodiment, provided herein is an amorphous form I of alogliptin benzoate which is characterized by an X-ray diffraction pattern having a single broad peak expressed between about 10 and about 35[20°]. The amorphous form I is further characterized by its glass transition temperature and by using various techniques including infrared absorption, Raman spectrometry, and thermal analysis (e.g. thermogravimetric analysis (TGA) and differential scanning calorimetry (DSC)).

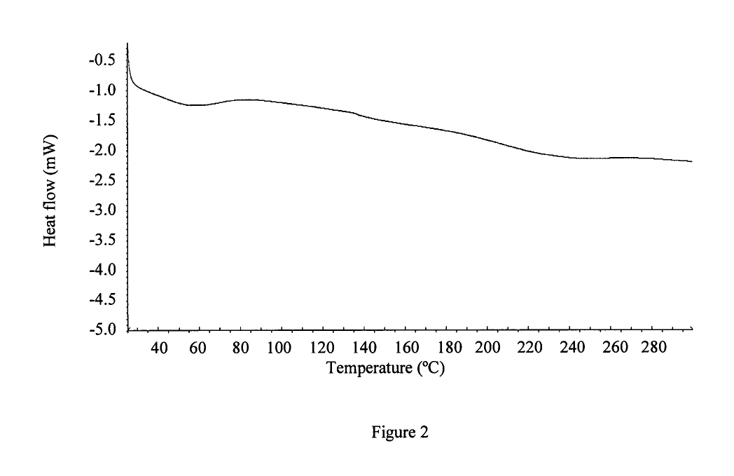

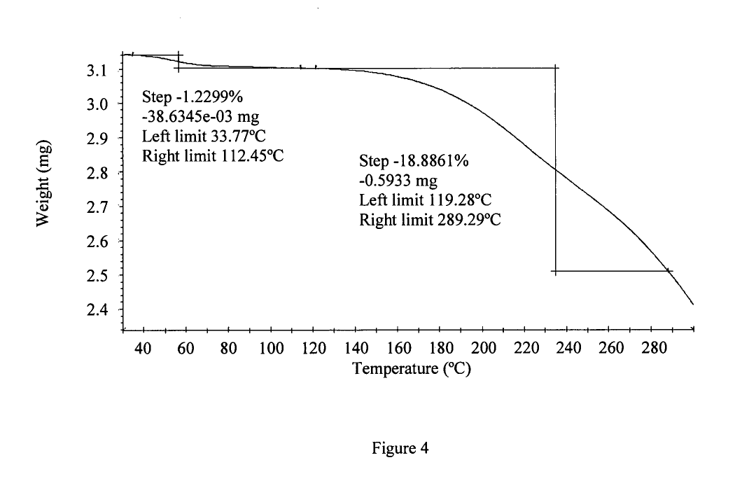

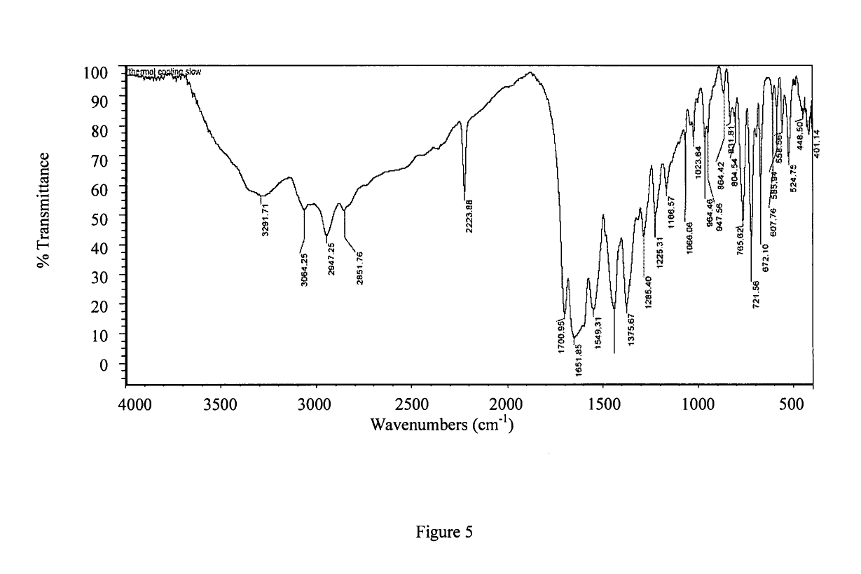

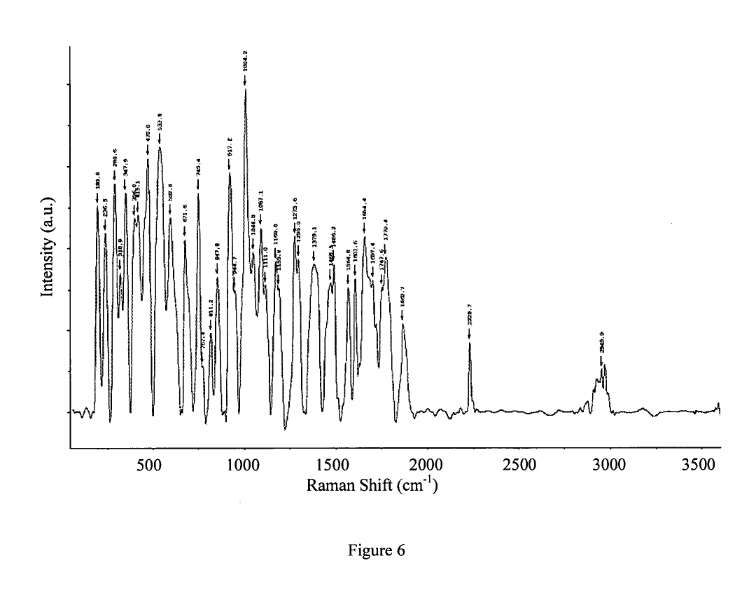

In some embodiments, the amorphous form I of alogliptin benzoate of the present invention is characterized by DSC and TGA profiles substantially as shown in FIGS. 2 and 4, respectively. In other embodiments, the amorphous form I is further characterized by modulate DSC to have glass transition temperature between about 66° C. and about 77° C. In other embodiments, the form is further characterized by an infrared spectrum substantially as shown in FIG. 5 with characteristic peaks at the following wavenumbers: about 401, about 448, about 525, about 559, about 586, about 608, about 672, about 722, about 766, about 805, about 832, about 864, about 948, about 964, about 1024, about 1066, about 1167, about 1225, about 1285, about 1376, about 1438, about 1549, about 1652, about 1701, about 2224, about 2852, about 2947, about 3064, and about 3292 cm⁻¹. In other embodiments, the amorphous form I of alogliptin benzoate is characterized by a Raman spectrum substantially as shown in FIG. 6 with characteristic peaks at the following wavenumbers: about 194, about 237, about 289, about 319, about 348, about 396, about 415, about 470, about 534, about 593, about 672, about 745, about 767, about 811, about 848, about 917, about 945, about 1004, about 1045, about 1087, about 1111, about 1170, about 1186, about 1274, about 1293, about 1379, about 1468, about 1486, about 1565, about 1602, about 1654, about 1697, about 1748, about 1770, about 1863, about 2229, and about 2950 cm⁻¹.

In other embodiments, the present invention further provides an amorphous form II of alogliptin benzoate which is characterized by an X-ray diffraction pattern having a single broad peak expressed between about 10 and about 35[29°]. The amorphous form II is further characterized by its glass transition temperature and by using various techniques including infrared absorption, Raman spectrometry, and thermal analysis (e.g. thermogravimetric analysis (TGA) and differential scanning calorimetry (DSC)).

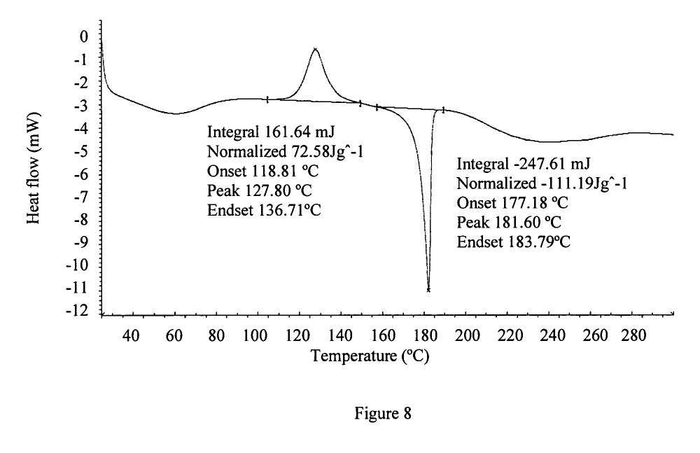

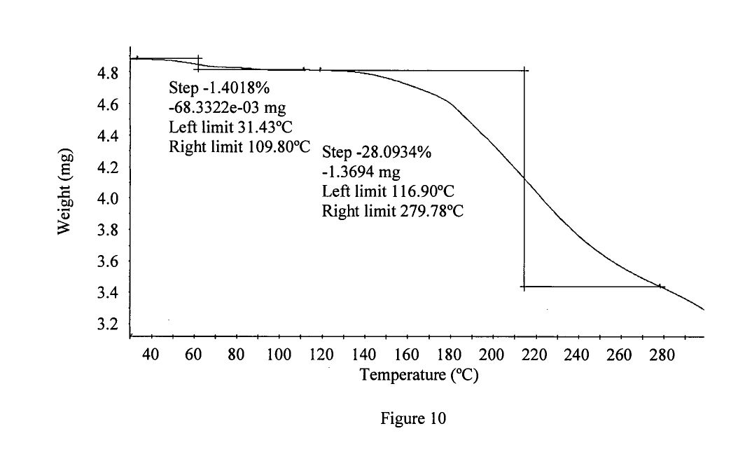

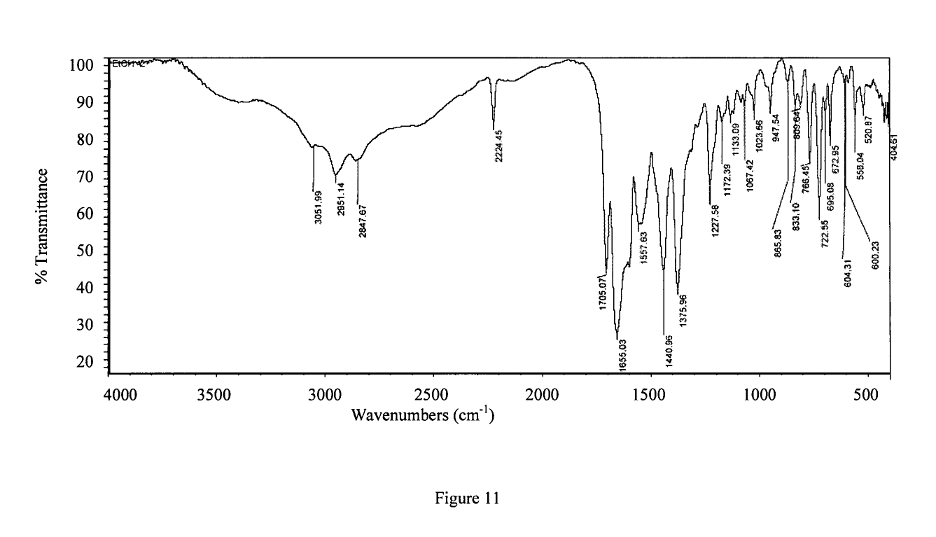

In some embodiments, the amorphous form II of alogliptin benzoate of the present invention is characterized by DSC and TGA profiles substantially as shown in FIGS. 8 and 10, respectively. In other embodiments, the amorphous form II is further characterized by modulate DSC to have glass transition temperature between about 68° C. and about 73° C. In some embodiments, the form is further characterized by an infrared spectrum substantially as shown in FIG. 11 with characteristic peaks at the following wavenumbers: about 405, about 521, about 558, about 600, about 604, about 673, about 695, about 722, about 766, about 810, about 833, about 866, about 948, about 1024, about 1067, about 1133, about 1172, about 1228, about 1376, about 1441, about 1558, about 1655, about 1705, about 2224, about 2848, about 2951, and about 3052 cm⁻¹.

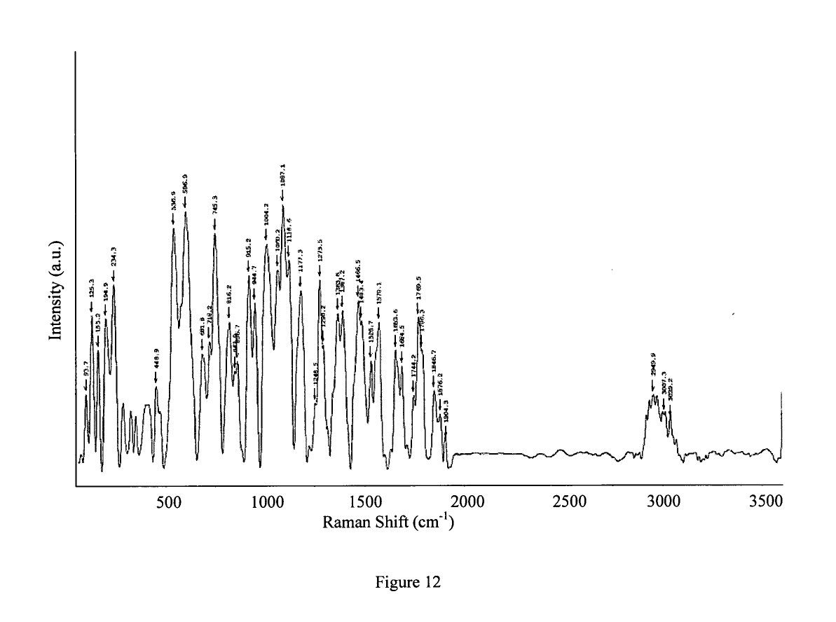

In other embodiments, the amorphous form II of alogliptin benzoate is characterized by a Raman spectrum substantially as shown in FIG. 12 with characteristic peaks at the following wavenumbers: about 94, about 125, about 155, about 195, about 234, about 449, about 537, about 597, about 682, about 718, about 745, about 816, about 843, about 857, about 915, about 945, about 1004, about 1060, about 1087, about 1119, about 1177, about 1248, about 1273, about 1290, about 1364, about 1387, about 1466, about 1483, about 1529, about 1570, about 1654, about 1685, about 1744, about 1769, about 1786, about 1847, about 1876, about 1904, about 2950, about 3007 and about 3039 cm⁻¹.

The present invention further provides processes from the preparation of the amorphous forms of the present invention. The processes include thermal precipitations and precipitations from supersaturated solutions. In particular, these processes involve the use of alogliptin benzoate, preferably alogliptin benzoate form A as the starting material or any other commercially available alogliptin benzoate or alogliptin benzoate prepared by any methods known in the art, including, for example, the methods described in EP 1586571 (WO 2005/095381) and in WO 2010/109468. The contents of all of the aforementioned references are hereby incorporated by reference in their entirety. Alternatively, alogliptin free base made in accordance with any method known in the art and converted to its benzoate salt by conventional methods can be used as the starting material in the processes of the present invention. According to one embodiment, the alogliptin benzoate starting material is heated until a melt is obtained, preferably under vacuum followed by controlled precipitation by slow/fast cooling. According to another embodiment, the alogliptin benzoate starting material is dissolved in a suitable solvent to prepare saturated solutions at room temperatures or at temperatures below the solvent boiling point. The solvent is then removed by evaporation.

Additional methods for the preparation of the amorphous forms of the present invention include, for example, precipitation from a suitable solvent, precipitation by cooling under vacuum, sublimation, growth from a melt, solid state transformation from another phase, precipitation from a supercritical fluid, and jet spraying. Techniques for precipitation from a solvent or solvent mixture include, for example, evaporation of the solvent, decreasing the temperature of the solvent mixture, freeze drying the solvent mixture, and addition of antisolvents (countersolvents) to the solvent mixture. The term “antisolvent” as used herein refers to a solvent in which the compound has low solubility.

Suitable solvents and anti-solvents for preparing the forms include polar and nonpolar solvents. The choice of solvent or solvents is typically dependent upon one or more factors, including solubility of the compound in such solvent and vapor pressure of the solvent. Combinations of solvents may be employed; for example, the compound may be solubilized into a first solvent followed by the addition of an antisolvent to decrease the solubility of the compound in the solution and to induce precipitation. Suitable solvents include, but are not limited to, polar aprotic solvents, polar protic solvents, and mixtures thereof. Particular examples of suitable polar protic solvents include, but are not limited to alcohols such as methanol, ethanol, and isopropanol. Particular examples of suitable polar aprotic solvents include, but are not limited to, acetonitrile, tetrahydrofuran (THF), dichloromethane, acetone, dimethylformamide, and dimethylsulfoxide.

The amorphous forms may be obtained by distillation or solvent addition techniques such as those known to those skilled in the art. Suitable solvents for this purpose include any of those solvents described herein, including protic polar solvents, such as alcohols (including those listed above), aprotic polar solvents (including those listed above), and also ketones (for example, acetone, methyl ethyl ketone, and methyl isobutyl ketone).

Non-limiting examples of the processes used to prepare each of the amorphous forms of the present invention are provided herein.

Methods for “precipitation from solution” include, but are not limited to, evaporation of a solvent or solvent mixture, a concentration method, a slow cooling method, a fast cooling method, a reaction method (diffusion method, electrolysis method), a hydrothermal growth method, a fusing agent method, and so forth. The solution can be a saturated solution or supersaturated solution, optionally heated to temperatures below the solvent boiling point. The recovery of the forms can be done for example, by filtering the suspension and drying. Alternatively, the solvents may be removed by rotary evaporation at desired temperatures.

The amorphous forms of the present invention can be prepared using fast/slow precipitation from saturated solutions in different solvents or mixture of solvents which are allowed to evaporate, preferably at room temperatures. Alternatively the saturated solutions can be heated followed by their cooling to induce precipitation as is known in the art.

The amorphous forms of the present invention can be also prepared by the slurry method as is well known in the art. Suspensions of the active ingredient in different solvents or mixture of solvents are prepared and shaken for long intervals (typically 24 hours).

Encompassed by the present invention are methods of antisolvent precipitation where an antisolvent is added to the saturated solution of the active ingredient in different solvents or mixture of solvents to induce precipitation.

Within the scope of the present invention are high pressure techniques where the active ingredient is compressed using various forces (e.g. grinding) as is known in the art.

As contemplated herein, the amorphous forms of the present invention can further be obtained using lyophilization wherein the compound is dissolved in water, followed by a freeze drying procedure.

The novel forms of the present invention are useful as pharmaceuticals for treating conditions mediated by DPP-IV. The present invention thus provides pharmaceutical compositions comprising any of the amorphous forms disclosed herein and a pharmaceutically acceptable carrier. The amorphous forms of the present invention can be safely administered orally or non-orally. Routes of administration include, but are not limited to, oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual. Typically, the amorphous forms of the invention are administered orally. The pharmaceutical compositions can be formulated as tablets (including e.g. film-coated tablets), powders, granules, capsules (including soft capsules), orally disintegrating tablets, and sustained-release preparations as is well known in the art.

Pharmacologically acceptable carriers that may be used in the context of the present invention include various organic or inorganic carriers including, but not limited to, excipients, lubricants, binders, disintegrants, water-soluble polymers and basic inorganic salts. The pharmaceutical compositions of the present invention may further include additives such as, but not limited to, preservatives, antioxidants, coloring agents, sweetening agents, souring agents, bubbling agents and flavorings.

Suitable excipients include e.g. lactose, D-mannitol, starch, cornstarch, crystalline cellulose, light silicic anhydride and titanium oxide. Suitable lubricants include e.g. magnesium stearate, sucrose fatty acid esters, polyethylene glycol, talc and stearic acid. Suitable binders include e.g. hydroxypropyl cellulose, hydroxypropylmethyl cellulose, crystalline cellulose, a-starch, polyvinylpyrrolidone, gum arabic powder, gelatin, pullulan and low-substitutional hydroxypropyl cellulose. Suitable disintegrants include e.g. crosslinked povidone (any crosslinked 1-ethenyl-2-pyrrolidinone homopolymer including polyvinylpyrrolidone (PVPP) and 1-vinyl-2-pyrrolidinone homopolymer), crosslinked carmellose sodium, carmellose calcium, carboxymethyl starch sodium, low-substituted hydroxypropyl cellulose, cornstarch and the like. Suitable water-soluble polymers include e.g. cellulose derivatives such as hydroxypropyl cellulose, polyvinylpyrrolidone, hydroxypropylmethyl cellulose, methyl cellulose and carboxymethyl cellulose sodium, sodium polyacrylate, polyvinyl alcohol, sodium alginate, guar gum and the like. Suitable basic inorganic salts include e.g. basic inorganic salts of sodium, potassium, magnesium and/or calcium. Particular embodiments include the basic inorganic salts of magnesium and/or calcium. Basic inorganic salts of sodium include, for example, sodium carbonate, sodium hydrogen carbonate, disodiumhydrogenphosphate, etc. Basic inorganic salts of potassium include, for example, potassium carbonate, potassium hydrogen carbonate, etc. Basic inorganic salts of magnesium include, for example, heavy magnesium carbonate, magnesium carbonate, magnesium oxide, magnesium hydroxide, magnesium metasilicate aluminate, magnesium silicate, magnesium aluminate, synthetic hydrotalcite, aluminahydroxidemagnesium and the like. Basic inorganic salts of calcium include, for example, precipitated calcium carbonate, calcium hydroxide, etc.

Suitable preservatives include e.g. sodium benzoate, benzoic acid, and sorbic acid. Suitable antioxidants include e.g. sulfites, ascorbic acid and a-tocopherol. Suitable coloring agents include e.g. food colors such as Food Color Yellow No. 5, Food Color Red No. 2 and Food Color Blue No. 2 and the like. Suitable sweetening agents include e.g. dipotassium glycyrrhetinate, aspartame, stevia and thaumatin. Suitable souring agents include e.g. citric acid (citric anhydride), tartaric acid and malic acid. Suitable bubbling agents include e.g. sodium bicarbonate. Suitable flavorings include synthetic substances or naturally occurring substances, including e.g. lemon, lime, orange, menthol and strawberry.

The amorphous forms of the present invention are particularly suitable for oral administration in the form of tablets, capsules, pills, dragées, powders, granules and the like. A tablet may be made by compression or molding, optionally with one or more excipients as is known in the art. Specifically, molded tablets may be made by molding in a suitable machine a mixture of the powdered active ingredient moistened with an inert liquid diluent.

The tablets and other solid dosage forms of the pharmaceutical compositions described herein may optionally be scored or prepared with coatings and shells, such as enteric coatings and other coatings well known in the art. They may also be formulated so as to provide slow or controlled release of the active ingredient therein using, for example, hydroxypropylmethyl cellulose in varying proportions to provide the desired release profile, other polymer matrices and the like. The active ingredient can also be in micro-encapsulated form, if appropriate, with one or more of the above-described excipients.

The present invention provides a method of treating a condition mediated by DPP-IV comprising administering to a subject in need thereof an effective amount of a composition comprising any one of the amorphous alogliptin benzoate forms of the present invention.

“A therapeutically effective amount” as used herein refers to an amount of an agent which is effective, upon single or multiple dose administration to the subject in providing a therapeutic benefit to the subject. In one embodiment, the therapeutic benefit is maintaining glucose homeostasis or regulating blood glucose levels. In additional embodiments, the amorphous forms of the present invention are used for the preparation of a medicament for treating conditions mediated by DPP-IV, preferably type 2 diabetes.

The present invention further provides the administration of the amorphous alogliptin benzoate forms in combination therapy with one or more other active ingredients. The combination therapy may include the two or more active ingredients within a single pharmaceutical composition as well as the two or more active ingredients in two separate pharmaceutical compositions administered to the same subject simultaneously or at a time interval determined by a skilled artisan.

The principles of the present invention are demonstrated by means of the following non-limiting examples.

### EXAMPLES

### Example 1

### General Preparation Methods of Alogliptin Benzoate Polymorphs

1. Reagents

Acetonitrile, HPLC grade, Sigma, Lot No. MKBC1316

Ethanol, AR, SCRC, Lot No. T10000418

DMSO, HPLC grade, Sigma, Lot No. 05737BH

Dichloride methane, AR, SCRC, Lot No, Lot No. 80047318

Methanol, AR, SCRC, Lot No. 80080418

Ethanol Acetate, AR, Yixing Secondary Chemical Company, Lot No. 090607

MIBK, AR, SCRC, Lot No. T20080411

Isopropyl alcohol, AR, Sinopharm Chemical Reagent Co. Ltd, Lot No. T20090813

Acetone, AR, Sinopharm Chemical Reagent Co. Ltd, Lot No. 10000418

Toluene, AR, SCRC, Lot No. T20090603

tert-Butyl methyl ether, HPLC grade, Fluka, Lot No. 1359496

THF, AR, Yixing Secondary Chemical, Lot No. 090901

1-Butanol, AR, SCRC, Lot No. T20080818

MEK, AR, SCRC, Lot No. T20090724

iPrOAc, AR, Shanghai Experimental Reagent Company, Lot No. 20080410

2-Me-THF, AR, Shanghai Jiachen Chemical Reagent Co. Ltd, Lot No. 090323

Heptane, HPLC grade, Sigma-Aldrich, Lot No. 05442LH

N-methylpyrolidone, HPLC grade, Sigma-Aldrich, Lot No. S86863-279

2. Instruments

Sartorius CP 225D Balance

ELGA Water Purification Equipment

Mettler Toledo DSC 1

Mettler Toledo TGA/DSC 1

Rigaku D/MAX 2200 X-ray powder diffractometer

Thermo Nicolet 380 FT-IR

Eyela FDU-1100 freeze dryer

Jobin Yvon LabRam-1B FT-Raman

3. XRPD, DSC, TGA and Microscope Methods

3.1 XRPD Method

Details of XRPD method used in the tests are mentioned below:
X-ray Generator: Cu, ka, (λ=1.54056 Á).
Tube Voltage: 40 kV, Tube Current: 40 mA.
DivSlit: 1 deg.
DivH.L.Slit: 10 mm
SctSlit: 1 deg.
RecSlit: 0.15 mm
Monochromator: Fixed Monochromator
Scanning Scope: 2-40 deg.
Scanning Step: 10 deg/min

3.2 DSC and TGA Methods

Details of DSC method used in the tests are mentioned below:
Heat from 25° C. to 300° C. at 10° C./min

Details of Modulated DSC method used in the tests are mentioned below:
Heat from 0° C. to 250° C. at 2° C./min, pulse height±1K

Details of TGA method used in the tests are mentioned below:
Heat from 30° C. to 300° C. at 10° C./min

3.3 FT-IR and FT-Raman Method
Details of FT-IR method used in the tests are mentioned below:
   No. of scan: 32
   Time for collection: 38 s
   Scan Range: 400-4000 cm⁻¹
   Resolution: 4

Details of FT-Raman method used in the tests are mentioned below:
Laser wave: 632.8 nm
Power: 1 mW
Resolution: 1 cm⁻¹
Time for integration: 50 s

4. General Preparation Methods

4.1 Method I: Thermal Heating/Cooling Experiments

Alogliptin benzoate form A of WO 2007/035372 (also referred to herein as alogliptin API) was heated to melt under vacuum followed by controlled precipitation of the melted compound by fast/slow cooling. Amorphous form I was identified by this method, as set forth in the Examples below.

4.2 Method II: Fast Precipitation from Saturated Solutions

Alogliptin benzoate form A of WO 2007/035372 (alogliptin API) was dissolved in ethanol at room temperatures to prepare saturated solutions. The ethanol was then removed using rotary evaporation below 50° C. Amorphous form II was identified by this method, as set forth in the Examples below.

### Example 2

### Amorphous Alogliptin Benzoate Form I (Method I)

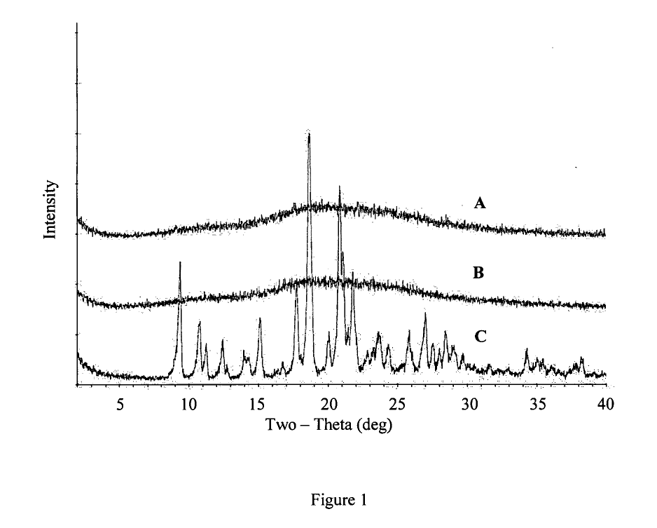

General method I was performed. Thus, alogliptin API was heated to 200° C. and then cooled down fast (quenching) or slow. This new polymorphic form showed a broad X-ray diffraction peak between about 10 and about 35 [2θ°] characteristic of an amorphous powder (FIG. 1, panels A and B). The amorphous phase was stable even after heating to 300° C. FIG. 2 illustrates a characteristic DSC profile. The DSC profile of the amorphous alogliptin benzoate form I of the present invention is significantly different from the DSC profile of the amorphous alogliptin benzoate disclosed in WO 2007/035372. For example, the amorphous alogliptin benzoate form I of the present invention shows a relatively smooth DSC profile with no exothermic peak at 132° C. and no endothermic peak at 183° C., contrary to the amorphous form 1 of WO 2007/035372. According to WO 2007/035372, recrystallization of the amorphous form 1 was recorded at 132° C., followed by the onset of the melt at 183° C. Thus the amorphous form 1 of WO 2007/035372 crystallized to crystalline form A during heating. In contrast, the amorphous form I of the present invention does not show these transitions in the DSC profile (FIG. 2). Without being bound by any theory or mechanism of action, the lack of sharp peaks in the DSC profile may indicate a more stable amorphous form.

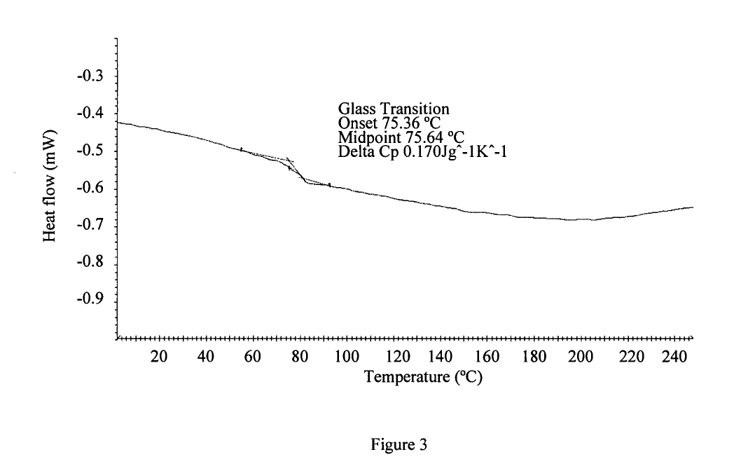

The amorphous form I of the present invention was further characterized by Modulated DSC in order to determine the glass transition temperature (FIG. 3). The glass transition temperature of the different batches is between about 66° C. and about 77° C. (variability is largely due to residual solvent effects). FIG. 4 illustrates a characteristic TGA profile: RT-120° C.—weight loss of 1.23%; 120° C.-290° C.—weight loss of 18.89%. FIG. 5 illustrates a characteristic IR spectrum with peaks at about 401, 448, 525, 559, 586, 608, 672, 722, 766, 805, 832, 864, 948, 964, 1024, 1066, 1167, 1225, 1285, 1376, 1438, 1549, 1652, 1701, 2224, 2852, 2947, 3064, and 3292 cm⁻¹. FIG. 6 illustrates a characteristic FT-Raman spectrum with peaks at about 194, 237, 289, 319, 348, 396, 415, 470, 534, 593, 672, 745, 767, 811, 848, 917, 945, 1004, 1045, 1087, 1111, 1170, 1186, 1274, 1293, 1379, 1468, 1486, 1565, 1602, 1654, 1697, 1748, 1770, 1863, 2229, and 2950 cm⁻¹.

### Example 3

### Amorphous Alogliptin Benzoate Form II (Method II)

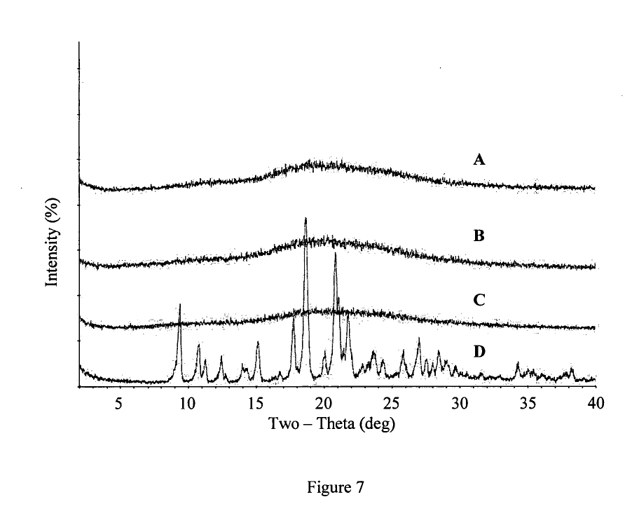

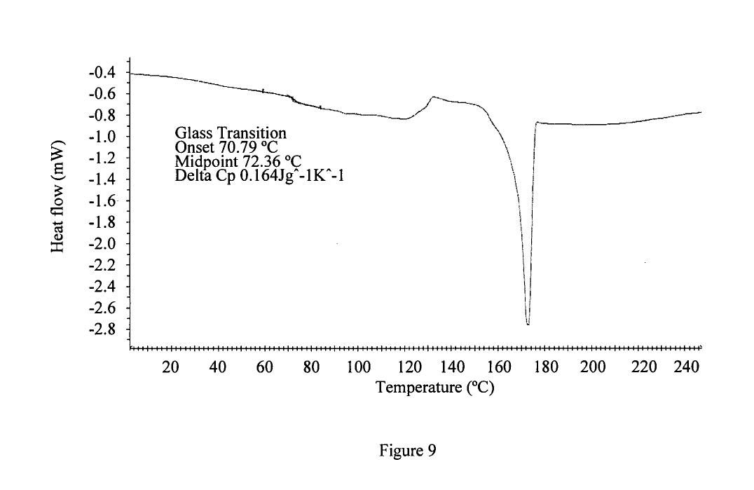

General method II was performed. Thus, alogliptin API was dissolved in EtOH at room temperatures until a saturated solution of alogliptin was obtained. The solvent was then evaporated using rotary evaporation below 50° C. This new polymorphic form showed a broad X-ray diffraction peak between about 10 and about 35 [2θ°] characteristic of an amorphous powder (FIG. 7, panel C). FIG. 8 illustrates a characteristic DSC profile having one exothermic peak at about 128° C. followed by an endothermic peak at about 182° C. The amorphous form crystallized to alogliptin API after the DSC measurement. The amorphous form II was further characterized by Modulated DSC in order to determine the glass transition temperature (FIG. 9). The glass transition temperature is between about 68° C. and about 73° C. (variability of the different batches is largely due to residual solvent effects). FIG. 10 illustrates a characteristic TGA profile: RT-120° C.—weight loss of 1.4%; 120° C.-280° C.—weight loss of 28.1%. FIG. 11 illustrates a characteristic IR spectrum with peaks at about 405, 521, 558, 600, 604, 673, 695, 722, 766, 810, 833, 866, 948, 1024, 1067, 1133, 1172, 1228, 1376, 1441, 1558, 1655, 1705, 2224, 2848, 2951, and 3052 cm⁻¹. FIG. 12 illustrates a characteristic FT-Raman spectrum with peaks at about 94, 125, 155, 195, 234, 449, 537, 597, 682, 718, 745, 816, 843, 857, 915, 945, 1004, 1060, 1087, 1119, 1177, 1248, 1273, 1290, 1364, 1387, 1466, 1483, 1529, 1570, 1654, 1685, 1744, 1769, 1786, 1847, 1876, 1904, 2950, 3007 and 3039 cm⁻¹. Raman spectroscopy revealed significant differences between the amorphous alogliptin benzoate form II of the present invention and the amorphous form 1 of WO 2007/035372, particularly at the 2000-3500 cm⁻¹ region (FIG. 12).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. An amorphous form of alogliptin benzoate (form I) characterized by a DSC profile substantially as shown in FIG. 2.

2. The amorphous form according to claim 1 having a glass transition temperature between about 66° C. and about 77° C.

3. The amorphous form according to claim 1 further characterized by a TGA profile substantially as shown in FIG. 4.

4. The amorphous form according to claim 1 further characterized by an IR spectrum substantially as shown in FIG. 5.

5. The amorphous form according to claim 4 wherein the IR spectrum has characteristic peaks at about 401±4, 448±4, 525±4, 559±4, 586±4, 608±4, 672±4, 722±4, 766±4, 805±4, 832±4, 864±4, 948±4, 964±4, 1024±4, 1066±4, 1167±4, 1225±4, 1285±4, 1376±4, 1438±4, 1549±4, 1652±4, 1701±4, 2224±4, 2852±4, 2947±4, 3064±4, and 3292±4 cm⁻¹.

6. The amorphous form according to claim 1 further characterized by a Raman spectrum substantially as shown in FIG. 6.

7. The amorphous form according to claim 6 wherein the Raman spectrum has characteristic peaks at about 194±4, 237±4, 289±4, 319±4, 348±4, 396±4, 415±4, 470±4, 534±4, 593±4, 672±4, 745±4, 767±4, 811±4, 848±4, 917±4, 945±4, 1004±4, 1045±4, 1087±4, 1111±4, 1170±4, 1186±4, 1274±4, 1293±4, 1379±4, 1468±4, 1486±4, 1565±4, 1602±4, 1654±4, 1697±4, 1748±4, 1770±4, 1863±4, 2229±4, and 2950±4 cm⁻¹.

8. A pharmaceutical composition comprising as an active ingredient the amorphous form of alogliptin benzoate (form I) according to claim 1 and a pharmaceutically acceptable carrier.

9. The pharmaceutical composition according to claim 8 in the form of a tablet.

10. (canceled)

11. (canceled)

12. A method of treating a condition mediated by DPP-IV comprising administering to a subject in need thereof an effective amount of a composition comprising the amorphous form of alogliptin benzoate (form I) according to claim 1.

13. The method according to claim 12 wherein the condition mediated by DPP-IV is type 2 diabetes and wherein the subject is a human.

14. (canceled)

15. A process for preparing amorphous alogliptin benzoate (form I) according to claim 1, comprising the steps of:
(a) heating a alogliptin benzoate to melt; and
(b) cooling the melted alogliptin benzoate obtained in step (a), so as to obtain amorphous alogliptin benzoate (form I).

16. The process according to claim 15, wherein the alogliptin benzoate is alogliptin benzoate Form A, or wherein the cooling step comprises fast cooling or slow cooling.

17. An amorphous form of alogliptin benzoate (form II) characterized by a Raman spectrum substantially as shown in FIG. 12.

18. The amorphous form according to claim 17, wherein the Raman spectrum has characteristic peaks at about 94±4, 125±4, 155±4, 195±4, 234±4, 449±4, 537±4, 597±4, 682±4, 718±4, 745±4, 816±4, 843±4, 857±4, 915±4, 945±4, 1004±4, 1060±4, 1087±4, 1119±4, 1177±4, 1248±4, 1273±4, 1290±4, 1364±4, 1387±4, 1466±4, 1483±4, 1529±4, 1570±4, 1654±4, 1685±4, 1744±4, 1769±4, 1786±4, 1847±4, 1876±4, 1904±4, 2950±4, 3007±4 and 3039±4 cm⁻¹.

19. The amorphous form according to claim 17 further characterized by a DSC profile substantially as shown in FIG. 8.

20. The amorphous form according to claim 17 having a glass transition temperature between about 68° C. and about 73° C.

21. The amorphous form according to claim 17 further characterized by a TGA profile substantially as shown in FIG. 10.

22. The amorphous form according to claim 17 further characterized by an IR spectrum substantially as shown in FIG. 11.

23. The amorphous form according to claim 22, wherein the IR spectrum has characteristic peaks at about 405±4, 521±4, 558±4, 600±4, 604±4, 673±4, 695±4, 722±4, 766±4, 810±4, 833±4, 866±4, 948±4, 1024±4, 1067±4, 1133±4, 1172±4, 1228±4, 1376±4, 1441±4, 1558±4, 1655±4, 1705±4, 2224±4, 2848±4, 2951±4, and 3052±4 cm⁻¹.

24. A pharmaceutical composition comprising as an active ingredient the amorphous form of alogliptin benzoate (form II) according to claim 17 and a pharmaceutically acceptable carrier.

25. The pharmaceutical composition according to claim 24 in the form of a tablet.

26. (canceled)

27. (canceled)

28. A method of treating a condition mediated by DPP-IV comprising administering to a subject in need thereof an effective amount of a composition comprising the amorphous form of alogliptin benzoate (form II) according to claim 17.

29. The method according to claim 28, wherein the condition mediated by DPP-IV is type 2 diabetes and wherein the subject is a human.

30. (canceled)

31. A process for preparing an amorphous alogliptin benzoate (form II) according to claim 17, comprising the steps of:
(a) dissolving alogliptin benzoate in ethanol; and
(b) evaporating the solvent to precipitate amorphous alogliptin benzoate (form II).

32. The process according to claim 31, wherein the alogliptin benzoate is alogliptin benzoate Form A.
